(19) United States
(12) Patent Application Publication
(10) Pub. No.: US 2022/0256939 A1
(43) Pub. Date: Aug. 18, 2022

(54) CUSTOMIZED KNITTED WEARABLE WITH REACTIVE MATERIAL FOR RIGIDITY

(71) Applicant: Aspen Medical Products, LLC, Irvine, CA (US)

(72) Inventors: Harry Duane Romo, JR., Aliso Viejo, CA (US); Frank Hernandez, Rancho Mission Viejo, CA (US); Joel Perez, Long Beach, CA (US)

(21) Appl. No.: 17/673,716

(22) Filed: Feb. 16, 2022

Related U.S. Application Data
(63) Continuation-in-part of application No. 17/178,071, filed on Feb. 17, 2021.

(51) Int. Cl. A41B 11/00; A41B 17/00; A41D 19/00

(52) U.S. Cl. CPC A41B 11/003; A41B 11/005; A41B 17/00; A41D 19/0006; A41B 2400/32; A41D 2500/50; A41B 2500/10; A41B 2500/50; A41D 2400/32; A41D 2400/38; A41D 2500/10; A41B 2400/38

(57) ABSTRACT

One embodiment relates to an orthopedic precast comprises a knitted shell portion and a knitted flexible portion. Heat or other hardening agent is used to harden the shell portion, while retaining flexibility in the flexible portion. Contemplated hardening agents include light, heat, and chemical polymerizing agents. In some embodiments the shell portion includes thermoplastic threads or yarns, which is then hardened by heating the thermoplastic sufficiently to at least partially melt, and thereby fuse together some of the threads or yarns, and then cooling to ambient temperature for enhanced rigidity. In other embodiments, the precast is contained in a bag or other airtight container, along with a self-heating composition that is triggered to release heat upon contact with oxygen. In still other embodiments, the precast includes a prepolymer of other polymerizable composition, which is polymerized by effective application of light, heat, and/or chemical agent(s) or deployed as a wearable.

### CROSS-REFERENCE TO RELATED APPLICATIONS

This application is a Continuation-in-Part of U.S. patent application Ser. No. 17/178,071 filed Feb. 17, 2021, the entire contents of which is incorporated by reference herein.

### FIELD

The field of the invention is orthopedic braces (orthoses).

### BACKGROUND

The following description includes information that may be useful in understanding the present invention. It is not an admission that any of the information provided herein is prior art or relevant to the presently claimed invention, or that any publication specifically or implicitly referenced is prior art.

Orthopedic braces (orthoses) usually need to be adjusted or customized in some manner to conform to the body part(s) being braced, and then properly positioned. A typical orthosis commonly has at least two portions, a rigid portion supporting a body part, and a flexible portion securing the orthosis to the body. The flexible portion is often a strap, and in many orthoses, multiple straps are required to adequately secure the orthosis. It can be time-consuming for a patient to repeatedly have to adjust the different straps.

U.S. Pat. No. 8,480,604 to Messer describes an ankle foot orthosis (AFO) which has a strap positioned around a calf region. Unfortunately, for some individuals a single strap might not be sufficient to secure the orthosis, given the complex ankle movements including dorsiflexion, plantarflexion, inversion, and eversion that occur during walking. Thus, the AFO could be mis-positioned during walking, providing a painful walking experience to the patient, and even potentially worsening a patient's medical condition.

U.S. Pat. No. 9,572,703 to Matthews describes an orthosis sock that utilizes a resilient material for restricting movement of a patient's foot. Because the orthosis is a sock, it is relatively easier to wear than a typical AFO. However, resiliency of the material can be insufficient to provide adequate support.

It is known to create custom AFOs by creating a negative mold of a patient's lower leg, ankle, and foot, using the negative mold to create a positive mold, wrapping preheated flexible and hardenable materials about different portions of the positive mold, and then applying vacuum to the material-wrapped positive mold then allowing time for the materials to cool to the shape of the positive mold. Once cooled, the materials must be carefully cut off the mold then all cut edges must be ground/smoothed to the final shape. Production of such custom AFOs is extremely labor intensive and inefficient. Production is time consuming, requires considerable skill, and is therefore relatively expensive.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, there is still a need for systems and methods for efficiently producing custom orthoses.

### SUMMARY OF EMBODIMENTS OF THE INVENTION

The inventive subject matter provides systems and methods in which a knitted wearable such as orthopedic precast/orthosis (hereinafter “precast”) or clothing item is described. According to one embodiment of the disclosure, the orthopedic precast features (i) a first knitted portion comprising one or more knitted strands of at least a first thermoplastic material that is mechanically coupled to (ii) a second knitted portion including lesser knitted strands of thermoplastic material (or different thermoplastic material) and/or including non-thermoplastic material. The first knitted portion and the second knitted portion may be attached as part of a single layer precast constructions, where these portions of the orthopedic precast utilize different material(s) and/or different knitting techniques to provide greater rigidity, maintain a flexible and/or elastic construction, or the like.

When heated to a prescribed temperature to melting of the first thermoplastic material, the first knitted portion transitions phase to become more rigid than its previous construction. This first knitted portion, sometimes referred to as a “shell portion,” may occupy different regions of the orthopedic precast. Whether the second knitted portion experiences a phase change depends on its material composition and structure. However, as described herein, the degree of phase change is different to provide regions of the orthosis with different levels of rigidity (e.g., rigid, flexible, semi-rigid, etc.).

As an illustrative example, for the situation where the second knitted portion includes the same thermoplastic material as the first knitted portion, but in lesser quantity (e.g., lesser strand numbers, thinner thermoplastic strands, lesser volume of thermoplastic strands due to knitting/stitching pattern, etc.), the second knitted portion may remain flexible (or at least less rigid) than the first knitted portion. Similarly, for the situation where the second knitted portion includes a second thermoplastic material that is different from and has a higher melting temperature than the first thermoplastic material, the second knitted portion may remain flexible where the heating temperature is below the melting temperature of the second thermoplastic material. Lastly, for the situation where the second knitted portion includes non-thermoplastic material, the second knitted portion may remain its flexible and perhaps elastic phase after the heating process to convert the orthopedic precast into an orthosis to be worn by a patient.

As used herein, the term “knitted” with respect to a portion of an object (e.g., orthopedic precast) is generally defined as any arrangement of material substantially comprising one or more strands (e.g., thread, yarn, etc.) to produce that portion of the object. This “knitting” may constitute an attachment of one or more types of materials together as a single layer of continuous material or multiple (two or more) layers of material through stitching (e.g., “V” shaped stiches), weaving (interlacing strands of material), crocheting (knot-like stitches), macramé (knot-like stiches in geometrical patterns), or another attachment scheme. Furthermore, the knitting may be in accordance with a two-dimensional (2D) knitting process or a three-dimensional (3D) knitting process. The 2D knitting process allows for the creation of a low-profile orthoses where the shell portion and flexible portion are knitted over a single knitted layer of material. The 3D knitting process may be utilized to create a layered knitted orthosis, such as added padding and/or additional layers of the same or different types of thermoplastic material may be added to the shell portion to increase its rigidity.

As used herein, the terms “rigid” or “rigidity” with respect to an object or portion of an object (e.g., a precast) means that the rigid object or rigid portion of the object will resist bending or deformation. According to this definition, different lengths of a given structure and composition can be rigid at a shorter length, and flexible at a longer length. As an illustrative example, in some cases, a “rigid” object or “rigid” portion(s) of the object may represent that the object or portion of the object may be deformed, in some cases permanently, if bent or twisted by at least 20° end to end.

As used herein, the term “resilient” with respect to an object or a portion of an object means that upon bending or stretching, the portion will automatically return to its substantially pre-bent ore pre-stretched shape. As used herein, the term “bending” may be construed to include twisting.

As used herein, the term “flexible” with respect to an object or a portion of an object means that the object or the object portion will not be permanently deformed by bending. For example, a knitted portion of a precast may be flexible in nature, even after a heating and cooling process. As used herein, the term “permanently deformed” means that deformation remains unless the deformation is actively repaired. According to this definition, an object or a portion of an object could be rigid in one direction, and flexible in another direction. Unless otherwise specified in such cases, the object or portion of the object is deemed to be rigid.

As used herein, the term “elastic” with respect to an object or portion of an object means that if the elastic portion is stretched or compressed lengthwise by at least 10%, it will return to its resting length, without the need for application of an external force, and without permanent deformation.

As used herein, the term “shell” means a structure configured to impart rigidity that restrains movement of a part of a patient's body, wherein the structure is either (i) rigid after an application of a prescribed heating and cooling phase or (ii) will become rigid upon completion of that heating and cooling phase. In particular, a knitted portion of a precast existing in a non-rigid may constitute a shell portion by having characteristics that allow this knitted portion to transition from its non-rigid form to a rigid form in response to an application of heat that is equal to or exceeds a prescribed melting temperature and subsequent cooling. In some embodiments, the shell may include a structure having a cavity, hollow, or lumen.

As used herein the term “patient” includes both humans and animals, independently of whether the patient is under the care of a medical or veterinary professional.

As used herein, the term “strand” is generally defined as an elongate, thin length of one or more natural, artificial, or combined natural and artificial substances. According to one embodiment of the disclosure, each strand may be a fiber no more than 3 millimeters (mm) thick over a length of at least one centimeter (cm), although other sizing is contemplated. Examples of a “strand” may include thread, yarn, string, cord, or any flexible material that may be organized into a structure (e.g., stitched, interwoven, etc.) to produce the object (e.g., orthopedic precast). Where the strand or stands denote thermoplastic material, these strands may be elongated fibers of the thermoplastic material, or alternatively, the strand may be another substance coated with thermoplastic material or impregnated with thermoplastic material.

In some embodiments, an orthopedic precast/orthosis may be configured to have a tube construction that includes both the shell and flexible portions. In some embodiments, the orthopedic precast/orthosis may be configured with a tube construction that includes shell portions of different levels of rigidity.

In some embodiments, the shell portion orients lengthwise along the tube. For example, the shell and flexible portions of a precast could correspond to the anterior and posterior portions of a lower leg respectively, and these portions can be directly connected to each other. For such a precast, the shell portion would be considered to be oriented lengthwise along the tube. Alternatively, a shell portion can orient crosswise with respect to a tube. For example, a precast capable of accommodating a torso portion of a patient could have a shell portion that extends across the front of a patient, and flexible portions that also extend across the front of the patient, connected superiorly and inferiorly to the shell portion. For such an orthopedic precast, the shell portion would be considered to be oriented crosswise along the tube.

As used herein, the term “crosswise” includes various degrees of diagonality.

A thermoplastic material used in a knitted (shell) portion can be different from or the same as a thermoplastic material used in an additional knitted (shell) portion. For example, the thermoplastic material used in a wrist part of a full arm wrist orthosis can be the same as or different from the thermoplastic material used in an elbow part. In preferred embodiments, the melting temperatures of different thermoplastic materials used in the same precast differ by 10°-20 C, by 10°-30° C., 30°-50° C., and even between 50°-150° C.

In some embodiments, the thermoplastic portion can comprise at least 30 wt % of a precast. In a preferred embodiment, the thermoplastic portion can comprise between 5 wt % and 90 wt % of the precast, more preferably between 50 wt % and 90 wt % of the precast, still more preferably between 80 wt % and 90 wt % of the precast.

Similarly, in some embodiments, the shell portion can comprise at least 30 wt % of a precast. In a preferred embodiment, the shell portion can comprise between 5 wt % and 90 wt % of the precast, more preferably between 50 wt % and 90 wt % of the precast, still more preferably between 80 wt % and 90 wt % of the precast.

In some embodiments, a shell portion of the precast can vary in nominal thickness by at least 50%. Similarly, a flexible portion of a precast can vary in nominal thickness by at least 50%.

In some embodiments, the flexible portion can be elastic. The elasticity can be achieved by a material itself having an elastic feature or knitting techniques being capable of having an elastic feature.

In some embodiments, the shell and flexible portion can be layered. For example, the flexible portion can be positioned adjacent to (e.g., laminated with) at least a layer of the shell portion to provide structural reinforcement. Alternatively, a shell portion can be positioned adjacent to (e.g., laminated with) at least one layer of a flexible portion to enhance skin comfort.

In some embodiments, a precast can include at least a formed aperture (eye) and a mating strap.

The inventive subject matter also includes methods of producing a custom orthosis, including the following operations:
1) Placing an orthopedic precast about a positive mold, the precast including (a) a first knitted portion operating as a shell portion that includes knitted strands of at least a first thermoplastic material that melts at a first melting point, and (b) a second knitted portion operating as a flexible portion that includes an elastic, knitted strands that do not melt below the first melting point; and
2) Heating the precast to at least 140° C. to partially melt and thereby fuse and rigidify the shell portion. Other contemplated minimum heating temperatures are set forth according to thermoplastics in the table below. One of ordinary skill in the art would appreciate that raising the temperature to completely melt a thermoplastic material would result in loss of functional shape of the shell portion, and therefore the processing temperature should be raised to only a lower part of the melting range. Also, the processing temperature should be raised at a speed at which the surface of the thermoplastic material has begun to melt, but the core of the thermoplastic material retains its shape. It is also contemplated that where there are different layers of thermoplastic material in the shell portion, the temperature parameters could be utilized such that the thermoplastic material in some of the layers melt more than the thermoplastic material in others of the layers. Experimentation has demonstrated that one successful method of implementing the inventive subject matter herein, is to provide a positive mold with through holes, and steam the orthopedic precast from the inside, through the positive mold.

Positive molds of a body part can be produced according to well-known techniques, including (a) using plaster or other materials to produce a negative cast of a body part, removing the negative cast from the body part, filing the cast with a hardenable casting material, and then removing the negative cast from about what is then the positive mold. It is also known to cut away or add material to the positive mold. Positive molds can be made from any body part or combination of adjoining parts, for example a positive mold could be made that mimics a hand, wrist and forearm. There are a number of advantage of placing a precast on a positive mold of a patient's body part upon which heat is applied to the precast generate the orthosis instead of a patient's body part. One advantage is that the mold may be reshaped from the patient's anatomical shape to add or remove certain contours that may create relief over a bony prominence of the patient or direct additional load to a certain soft tissue area that can handle the additional load in order to increase the comfort of the patient when the orthosis is worn.

In preferred methods the precast may be somewhat tubular, with one or two open ends so that the precast can be pulled over a body part. In some embodiments a knitted portion operating as the shell portion may include multiple types of thermoplastic material, where the second or third thermoplastic materials would have a different melting point from a melting point of a first thermoplastic material.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a perspective view of an exemplary embodiment of a precast for a torso orthosis.

FIG. 2 is a perspective view of a first exemplary embodiment of a precast of a leg-ankle-foot orthosis, according to inventive principles herein.

FIG. 3A is a perspective view of a second exemplary embodiment of precast of a leg-ankle-foot orthosis, according to inventive principles herein.

FIG. 3B is a vertical cross sectional view of the precast of FIG. 3A.

FIG. 4 is a perspective view of a third exemplary embodiment of a precast of a leg-ankle-foot orthosis, according to inventive principles herein.

FIG. 5 is an exemplary embodiment of perspective views of dorsal and palmar views of a left person's left hand, wearing a precast of a wrist orthosis, according to inventive principles herein.

FIG. 6 shows a heating process of an embodiment of a precast, according to inventive principles herein.

FIG. 7A is a schematic of an exemplary embodiment of an orthopedic precast, as for example in the precasts of FIG. 1, 2, 4, 5, or 6.

FIG. 7B is a schematic of the exemplary orthopedic precast portion of FIG. 7A, in which the thermoplastic material is heated.

FIG. 8A is a schematic of an exemplary embodiment of a portion of an orthopedic precast having a first knitted portion configured with a higher concentration of a thermoplastic material than in a second knitted portion.

FIG. 8B is a schematic of an exemplary embodiment of a portion of an orthopedic precast having a first knitted portion configured with a tighter knit than in a second knitted portion.

FIG. 8C is a schematic of an exemplary embodiment of a portion of an orthopedic precast having a first knitted portion configured with thicker strands of a thermoplastic material than in a second knitted portion.

FIG. 8D is a schematic of an exemplary embodiment of a portion of an orthopedic precast having a first knitted portion configured with a higher number of layers that include filaments of a thermoplastic material than in a second knitted portion.

FIG. 9 is a schematic of an exemplary embodiment of a portion of an orthopedic precast having a strap.

FIG. 10A is a schematic of an exemplary embodiment of a portion of an orthopedic precast in which a first knitted portion is sewn or knitted to a second portion.

FIG. 10B is a schematic of an exemplary embodiment of a portion of an orthopedic precast in which a first knitted portion is laminated to a second portion.

FIG. 10C is a schematic of an exemplary embodiment of a portion of an orthopedic precast in which a first knitted portion is chemically bonded to a second portion.

FIG. 10D is a schematic of an exemplary embodiment of a portion of an orthopedic precast in which a first knitted portion is partially melted and thereby fused to a second portion.

FIG. 11 is a perspective view of an exemplary embodiment of a portion of an orthopedic precast in which a first knitted portion is coupled with a second, elastic portion.

FIG. 12A is a perspective view of an exemplary embodiment of an orthopedic precast sleeve in which a first knitted portion is layered with a second knitted portion.

FIG. 12B is a perspective view of the precast of FIG. 12A, in which a top edge portion of the second knitted portion of the precast sleeve has been folded down over at least part of the first knitted portion.

FIG. 13 is a perspective view of a portion of an orthopedic precast having a strap that extends through an aperture formed in the first knitted portion.

FIG. 14A is a perspective view of an exemplary embodiment of an orthopedic precast positioned about an inanimate mold.

FIG. 14B is a perspective view of the orthopedic precast of FIG. 14A positioned about the lower leg, ankle and foot of a mold of a patient to produce an ankle-foot-orthosis (AFO) derived from the orthopedic precast.

FIG. 15 is a perspective view of an exemplary embodiment of a self-heating, orthopedic precast disposed in non-oxygen filled bag to cause an exothermic reaction, having embedded self-heating material.

FIG. 16 is a perspective exploded view of an alternative embodiment of a self-heating, orthopedic precast disposed in a nitrogen filled bag, having self-heating material in an outer cover.

FIG. 17 is a perspective view of an exemplary embodiment of an alternative precast, which includes a polymerizable material. The precast is stored in a bag that excludes a polymerizing agent.

FIG. 18 is a perspective view of an exemplary embodiment of a shoulder orthosis produced by a precast featuring one or more first knitted portions with thermoplastic material.

FIG. 19 is a perspective view of an exemplary embodiment of a post-operative shoe configured with the first knitted portion positioned along a foot plate and selected areas surrounding an ankle area.

FIG. 20 is a perspective view of an exemplary embodiment of a clothing item with stands of thermoplastic material knitted to form one or more channels within the clothing item.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities of ingredients, properties such as concentration, reaction conditions, and so forth, used to describe and claim certain embodiments of the invention are to be understood as being modified while still retaining the spirit and scope of the disclosed invention. Accordingly, in some embodiments, the numerical parameters set forth in the written description and attached claims are illustrative approximations that can vary depending upon the desired properties sought to be obtained by a particular embodiment.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

I. Illustrative Orthosis Architecture

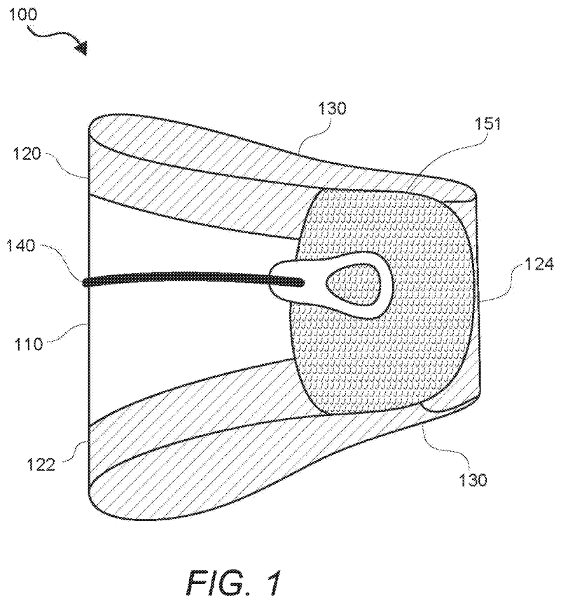

FIG. 1 generally depicts an orthosis 100 generated from a precast, sized and dimensioned to wrap about a torso of a patient and operate as a lumbar sacral orthosis (LSO) or part of a thoracic lumbar sacral orthosis (TLSO). The orthosis 100 may be configured with a first portion 110 knitted with one or more strands comprising thermoplastic material, which features a rigid construction after a prescribed amount of heat is applied to the first portion 110 (referred to as a “shell portion”). Featured above and below the shell portion 110, upper and lower portions 120 and 122 may be knitted with one or more strands comprising non-thermoplastic material (each referred to as a “flexible portion”). As shown, for this embodiment of the disclosure, the shell portion 110 and the flexible portions 120, 122 generally compose a tube (e.g., belt) 130, with two open ends. In this particular example, both the shell portion 110 and the flexible portions 120, 122 are oriented crosswise with respect to the tube 130.

As shown in FIG. 1, the flexible portions 120, 122 is positioned on an outer edge of the belt 130 to create a transitional area between the rigid shell portion 110. This transitional area provides a more comfortable fit of the orthosis 100, while at the same time, creates greater anatomical stabilization from placement of the shell portion 110 within the orthosis 100. Also, the integration of the non-thermoplastic material of the flexible portions 120, 122 (positioned along the edges of the orthosis 100) and the thermoplastic material forming the shell portion 110 (interposed there between) allows for the construction of a low-profile (single layer) orthosis in lieu of a multi-layer orthosis with hardened panels attached at different segments of the belt 130.

Herein, according to one embodiment of the disclosure, each strand of the one or more strands forming the shell portion 110 may include at least one type of thermoplastic material. For example, the strand(s) forming the shell portion 110 may include a single type of thermoplastic material. Alternatively, the strand(s) forming the shell portion may constitute a composite, which may include at least two different thermoplastic materials, where the thermoplastic materials may have the same or different melting temperatures. As yet another alternative embodiment of the disclosure, the strand(s) can also be a composite of one or more thermoplastic materials and one or more non-thermoplastic materials, provided the rigidity of the shell portion 110 is altered after a selected temperature and duration of heat is applied and the shell portion 110 is cooled. The strand(s) may be formed from the thermoplastic material(s) or the strand(s) of a different material may be coated and/or impregnated (through an application process) with one or more thermoplastic materials. Herein, “thermoplastic material” may reference a collection of material inclusive of one or more strands of thermoplastic material that, when heated and cooled, alter the overall rigidity of the material while “non-thermoplastic material” is devoid of thermoplastic material to affect such rigidity.

According to one embodiment of the disclosure, the thermoplastic materials may be configured to form flexible strands at room temperature, are non-toxic, melt between 140° C. and 350° C., and become rigid when strands are partially melted together into a sheet or mat having a thickness of 0.5 mm to 6 mm. Contemplated examples of thermoplastic materials may include, but are not limited or restricted to the following: Polyethylene Terephthalate (PET), Polyether ether ketone (PEEK), Polyphenylene oxide (PPO), Polypropylene (PP), polyethylene (PE), polyvinyl chloride (PVC) and polystyrene (PS), poly(methyl methacrylate) (PMMA), Acrylonitrile butadiene styrene (ABS), Polylactic acid (PLA), Polybenzimidazole (PBI), Polycarbonate (PC), Polyether sulfone (PES), Polyoxymethylene (POM), Polyphenylene sulfide (PPS), Polystyrene, Polyvinyl chloride (PVC), Polyvinylidene fluoride (PVDF), Polytetrafluoroethylene (PTFE), Polyamide 6 (PA6), Polybutylene terephthalate (PBT), Polyetherimide (PEI), or the like.

The thermoplastic and non-thermoplastic materials can be selected using any combination of natural and synthetic materials to accomplish a desired characteristic, as for example, a desired degree of stiffness, compressibility, flexibility, bending, stretch, and resilience. As described above, one or more strands associated with the rigid shell portion 110 of the orthosis 100 may include both thermoplastic and non-thermoplastic materials. For example, the non-thermoplastic material may include Kevlar™ to increase the durability/toughness of the orthosis 100, as one or more other non-thermoplastic materials (e.g., cotton fibers, non-carbon fibers, nanotubes, glass fibers, ceramic, and/or metal fibers) may be used. Similarly, one or more strands of material forming the flexible portion(s) 120 and/or 122 can also comprise thermoplastic and/or non-thermoplastic materials. However, according to this embodiment of the disclosure, the thermoplastic material of the flexible portion(s) 120 and/or 122 may feature a lowest melting point that is substantially above the lowest melting point of the thermoplastic material used in the shell portion 110.

As a result, one of the inventive concepts is that a precast forming the orthosis 100 will have (1) one or more knitted strands of a first (thermoplastic) material or set of materials, which upon heating, partially melt and therefore fuse together to form the rigid shell portion 110, and (2) one or more knitted strands of a second (non-thermoplastic) material or set of materials that remain flexible upon cooling, either because they do not melt, or they melt to an insubstantial amount at the temperature used to melt the thermoplastic materials forming the shell portion 110, collectively form the flexible portions 120 and 122. Accordingly, the terms “insubstantial” and “substantial” are used herein in that context as to the amount of thermoplastic material melting to change a portion of the precast of the orthosis into a rigid state.

It should be appreciated then, that the one or more knitted strands of a different material or materials that remain flexible upon cooling might or might not include a thermoplastic material. Preferably, however, the one or more knitted strands of a different material or materials that remain flexible upon cooling could mostly or entirely comprise a natural fiber such as cotton or wool. To avoid oxidation of such non-thermoplastic materials, heating can take place in an anoxic or low oxygen environment.

In production, a precast associated with the orthosis 100 may be placed over a positive mold and heated, such that at least some of the thermoplastic material(s) fuse, in what will become a rigid shell. This allows the shell portion 110 to closely conform to whatever part(s) of the patient are to be motion-restrained. Alternatively, the precast may be placed over a first positive mold, removed inside-out to provide a different (opposite) layering scheme for this flipped precast, which is placed over a second positive mold, heated, and subsequently cooled to form an orthosis with a rigid shell portion or an orthosis with multiple layers when the flipped precast is arranged with another precast.

The precast of the orthosis 100 has a tubular configuration, with superior and inferior open ends. However, as depicted in FIG. 1, the orthosis 100 can also open and close laterally, using a fastener such as a Velcro™ or similar hook and loop fastener 151, which can be installed at the precast stage. Any suitable fasteners are contemplated, including buttons, toggles, studs, snap fastener, poppers, buckles, zippers, frogging, hooks and eyes, magnets, grommets, brooches, safety pins, fabric ties, and laces.

In FIG. 1, the flexible portions 120, 122 can advantageously be elastic, and in especially preferred embodiments, portions 120, 122 can be knitted to be increasingly flexible and/or elastic towards the outer edges. Such variance in flexibility and elasticity can promote the patient's comfort by transitioning pressure against the body.

The flexible portions 120 and 122 are preferably elastic even after the heating and cooling processes. Elasticity is advantageous because it causes the orthosis 100 to conform to different body shapes. Moreover, since the shell portion 110 of the orthosis 100 has limited extendibility (e.g., extends partway around the precast), the flexibility of portion 124 could be sufficient to allow a user to pull the orthosis 100 around the waist, as an alternative means of placement.

The orthosis 100 includes a strap or cord 140 to further secure the orthosis 100 on a patient's body, such as by cinching mechanism (not shown) positioned along a backside of the precast 100.

II. Illustrative Preset Architectures

The following illustrations and description is directed to precast architectures that may be used to generate resultant orthoses with the same architecture. The illustrative architectures are not limited, but rather, have been selected to highlight functionality that may be deployed in any orthosis architecture.

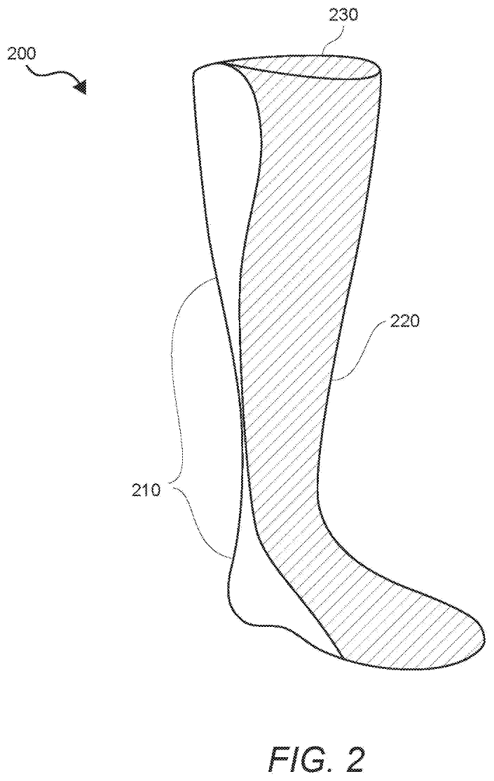

FIG. 2 generally depicts an orthopedic precast 200, which can be heated and cooled to produce a first type of ankle-foot-orthosis (AFO) configured to restrict movement of lower leg of a patient relative to the foot. The precast 200 generally includes a first knitted portion 210 and a second knitted portion 220 generally configured as a long sock featuring a tube 230 having an open upper calf end and a closed toe end. The second knitted portion 220 may be formed with elastic, non-thermoplastic material operating as a flexible portion for the precast 200 (as well as the resultant orthosis) while the first knitted portion 210 may be formed with thermoplastic material operating as the shell portion for the precast (and resultant orthosis). The precast 200 could also be provided in a preformed condition, in an average shape of a given anatomical size. Various sizes could be offered accordingly. This pre-shaped item could be commercially offered as an “off-the-shelf” product that could be provisioned to a patient of average contours given their dimensions without modification. It could also provide the opportunity for optimization of the contours through heating and reforming the shell material(s) in strategic locations.

In a corresponding precast 200, the first knitted portion 210 and the second knitted portion 220 cooperate to support posterior and anterior parts of the lower leg, respectively. The first knitted portion 210 and the second knitted portion 220 are oriented lengthwise along the tube 230. In this configuration, the second knitted (flexible) portion 220 allows easy on-off of a corresponding orthosis, while the first knitted (shell) portion 210 provides dorsiflexion, plantarflexion, inversion, and eversion stability at the ankle.

Strategic use of elastic regions can enhance functionality. For example in FIG. 2, the second knitted (flexible) portion 220 can be elastic, and such elasticity can function to press the first knitted (shell) portion 210 against the back of the leg, and that can assist in lifting the foot during swing phase of ambulation. In some embodiments, an orthosis derived from the precast 200 can have a slightly dorsiflexed configuration such that when worn, the weight of the foot pulls the foot into a neutral (neither dorsiflexed nor plantarflexed) or other desired configuration.

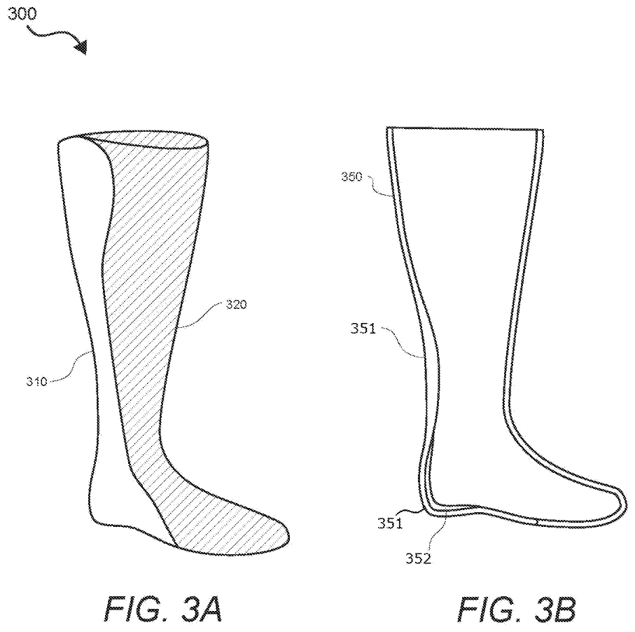

FIG. 3A generally depicts a precast 300 of second AFO type. The first knitted (shell) portion 310 of the precast 300 is wider (encompassing a greater amount of circumference) and potentially thicker than the first knitted (shell) portion 210 of FIG. 2. This provides relatively greater (sagittal plane and coronal plane) stability to the ankle than the AFO precast 200 of FIG. 2, which provides ancillary assistance in more stability of the knee of the patient.

During the knitting process, the relative dimensions of the shell portion 310 and flexible portion 320, the knitting patterns utilized by each of these portions 310/320, and areas of different thicknesses, can be easily customized, among other things to provide reinforcement where desired. For example, different precasts, similar to the precast 300, may be configured with shell portions (e.g., shell portion 310) with different attachments patterns (e.g., knitting inclusive of stitching, weaving, etc.) that alters their rigidity such as different regions of the shell portion 310 having different rigidity levels due to these regions using different knitting patterns or different thermoplastic strand thickness as described below. Hence, the precast 300 may feature the shell portion 310 with a certain rigidity level while the shell portion of another precast may be configured with a more or less rigidity after heating and cooling.

In FIG. 3B, for example, region 351 is knitted to be thicker than region 350. Thickness(es) can be customized by using different thermoplastic materials, different types or thicknesses of strands, and/or varying the knitting technique. Herein, shell portions and flexible portions can be coupled in any suitable manner, including lateral juxtaposition and overlapping (e.g., a flexible portion 351 inside and layered or laminated against an area of the shell portion 352).

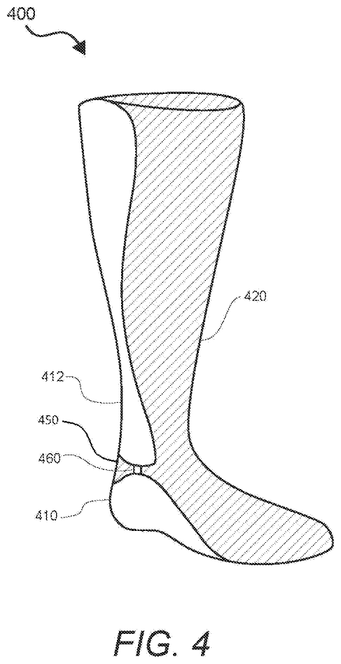

FIG. 4 generally depicts a precast 400 corresponding to an articulated AFO. Relative to orthoses generated from the precasts 200, 300 of FIGS. 2-3, an orthosis generated from the precast 400 would accommodate greater ankle movement, greater medial lateral stability, and greater motion along the sagittal plane.

Herein, according to this embodiment of the disclosure, the precast 400 includes a plurality of knitted portions; namely, a first knitted portion 410 operating as a lower shell portion (foot plate section) and a second knitted portion 412 operating as an upper shell portion (calf section) for this example. These shell portions 410 and 412 are at least partially coupled by a third knitted portion 420 operating as a first flexible portion and/or a fourth knitted portion 450 operating as a second flexible portion. The second flexible portion 450 is arranged to separate the lower shell portion 410 from the upper shell portion 412 within a resultant orthosis. The second flexible portion 450 may be knitted in as part of the precast 400 or may constitute a post-production element for attachment at a connection point 460 (and optionally a second connection point (not shown) positioned on an opposite side of the ankle). The attachment may be accomplished, for example, by a fastener positioned at each connection point.

According to one embodiment of the disclosure, the second flexible portion 450 may be elastic to provide different degrees to tension to the Achilles tendon of the patient. Alternatively, the second flexible portion 450 may be flexible, but may be inelastic or may feature one or more inelastic connection points 460 at which the second flexible portion 450 is attached to the first flexible portion 420.

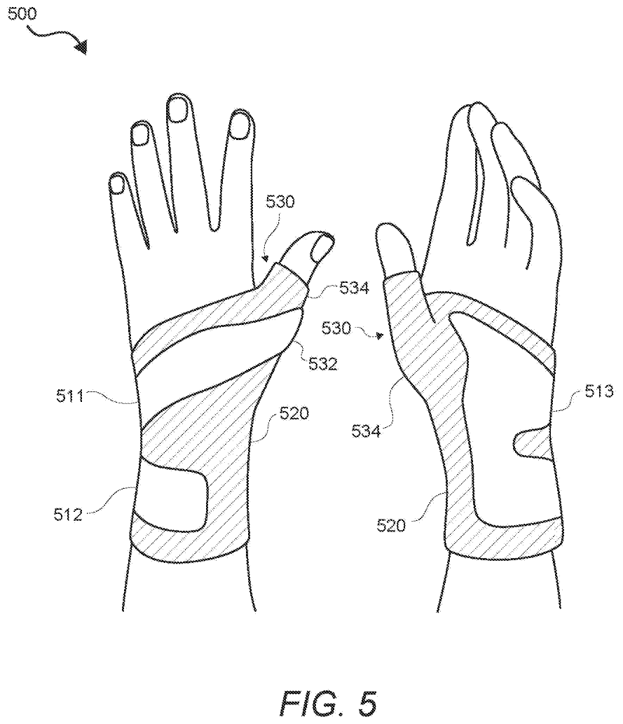

FIG. 5 generally depicts dorsal and palmar views of a left person's left hand, wearing a precast 500 of a wrist orthosis. The shell portion appears as two physically separated regions 511, 512 on the dorsal side, but a single region 513 on the palmar side. The flexible portion 520 extends entirely around the shell portions 511, 512, and 513. On the orthosis for each hand, there are three openings, one for the thumb, one for the wrist, and one for the fingers/distal metacarpal region. In this particular example, the thumb spica 530 includes a shell portion 532 and a flexible portion 534.

A wrist orthosis corresponding to precast 500 would be effective in reducing wrist flexion, extension, abduction, adduction and rotational movement, while still being relatively easy to put on because of the flexible portion 520. The rigidity of the shell portions 511, 512, and 513 may be the same, or the rigidity between these shell portions 511, 512, and 513 may differ based on the type of thermoplastic material used, number and/or concentration of strands of the thermoplastic material within these shell portions, the knitting pattern utilized, etc.

One advantage associated with this type of wrist orthosis is that the patient would slide the orthosis on as a glove with no tightening strap(s) required. With integrated regions of rigidity, the wrist orthosis is configured to stabilize the wrist joint and/or the thumb without additional latching of straps or other fasteners.

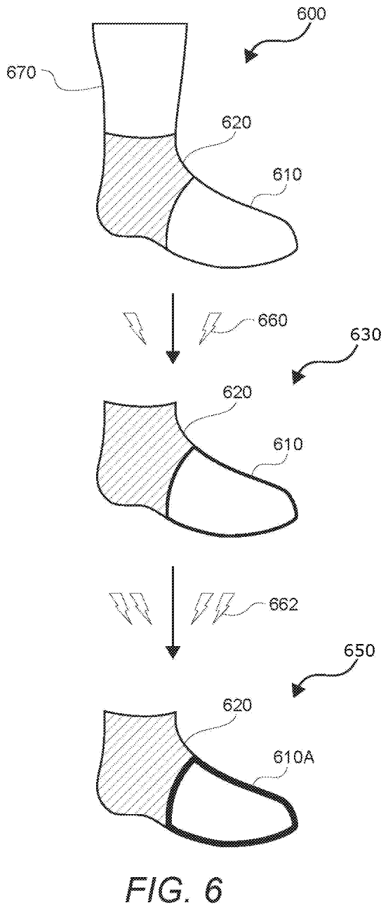

FIG. 6 generally depicts stages in production of a pre-fabricated orthosis from a knitted precast 600. As shown, the precast 600 is directed to a foot orthosis designed to protect a front foot area of a patient, albeit the development of the pre-fabricated orthosis may be directed to any part of a patient's body. Herein, the precast 600 includes knitted strands having at least two different materials 610 and 620. For example, the different materials 610 and 620 may include thermoplastic material and non-thermoplastic material, respectively. Alternatively, the different materials may include a first thermoplastic material 610 having a lower melting temperature than a second thermoplastic material 620.

Herein, the precast 600 may be placed on a positive mold 670 of a targeted body part, and heated to a first temperature 660 (e.g., a lower melting point temperature) that partially melts the thermoplastic material 610. Once cooled, this provides sufficient stiffness so that the precast 600 can retain its shape when removed from the positive mold (or patient). After removal, the stiffened precast 630 may be provided or sold to a clinician as a pre-fabricated orthosis.

Thereafter, upon receipt by the clinician, the stiffened precast 630 may be placed on a mold associated with a body part of the patient, hand molded as desired, and heated to a second temperature 662. The second temperature 662 may be greater than the first temperature 660 to allow for reshaping of the first thermoplastic material 610. Once cooled, the precast 650 becomes a customized orthosis, with now-hardened shell portion 610A located in a first region of the orthosis.

Although not shown, where the precast 600 includes knitted strands with the first thermoplastic material 610 having a lower melting temperature than the second thermoplastic material 620, the stiffened precast 630 may be placed on a mold of the patient, hand molded as desired, and heated to the second temperature 662. The second temperature 662 may be greater than the first temperature 660 to allow for reshaping of the first thermoplastic material 610 as well as melting of at least a portion of the second thermoplastic material 620. Once cooled, the precast 650 becomes a customized orthosis, with the hardened shell portion 610A along with another hardened shell portion positioned in a second region (e.g., ankle-heel region).

As an alternative embodiment, the precast 600 may be placed on a body part of a patient in lieu of the positive mold. Herein, the first temperature 660 would fall within a temperature range that would not injure the patient, where a series of heating processes may occur to allow a clinician to adjust and mold during stiffening phases of the precast 630 until the precast 650 becomes a customized orthosis.

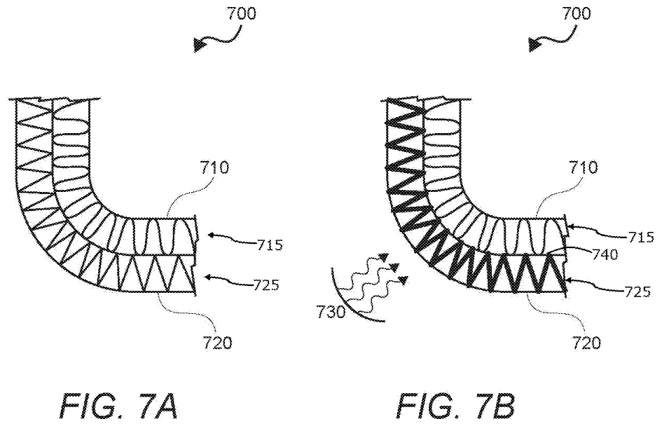

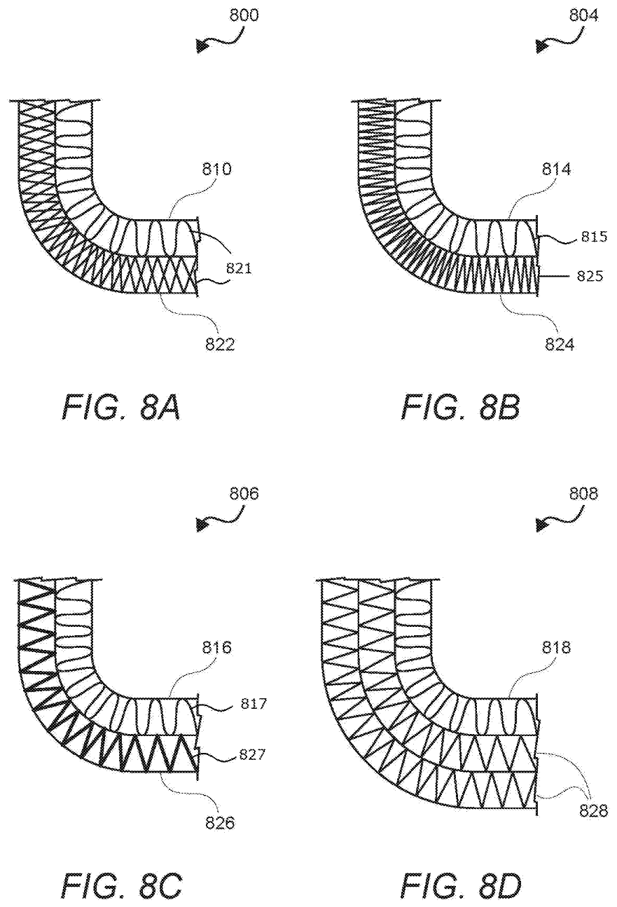

Referring now to FIGS. 7A-8D, embodiments of precasts with multiple layers of knitted material are described. According to these embodiments, each precast may include multiple knitted layers of materials. Precasts with multiple material layers may be accomplished through a number of techniques, including complex multi-layering knitting processes, separate knitted portions positioned on each other and attached together, or a precast with a single layer of knitted materials that is folded over itself or another precast to form multiple layered knitted portions. As an illustrative example, for a precast formed for a body part (e.g., foot, leg, hand, or arm), the first knitted portion may be directed to a first body part (e.g., right foot) and a second knitted portion may be directed to a complementary second body part (e.g., left foot) arranged facing opposite to and in-line with the first body part. A knitting process is undertaken with a first sock arrangement knitted with a first material (e.g., starting with open tubular area and concluding at a closed-ended tubular area knitted for a right foot facing a first direction) continued with a second sock arrangement knitted with a second material (e.g., closed-ended tubular area knitted for a left foot facing a second direction opposite the first direction continuing to an open tubular area). Thereafter, the second sock arrangement is folded around itself to envelop the first sock arrangement so as to produce a sock orthosis precast with multiple layers including an interior first material and an external second material.

FIG. 7A generally depicts a schematic of an embodiment of an orthopedic precast 700 including a second knitted portion 720 featuring one or more strands of a second material 725 with a lower melting point than one or more strands of a first material 715 associated with a first knitted portion 710. For this illustrative embodiment, different materials 715 and 725 may be layered when forming the precast 700. For example, the second material 725 may be partially melted when a prescribed temperature is applied to provide rigidity after cooling while the first material 715 may experience no phase change (e.g., no change from solid to partial liquid phase that is cooled to re-solidify), but instead, operates as an inner spacer to provide padding and airflow.

As with all examples herein, where the second material 725 associated with the second knitted portion 720 constitutes a thermoplastic material with a lower melting point than the first material 715 associated with the first knitted portion 710, it is contemplated that the difference in melting points can arise because the first material 715 has no melting point or a melting point considerably greater than the melting point of the second material 725. For example, the first material 715 may be a nylon or Kevlar™ for example.

FIG. 7B generally depicts a schematic of the portion of an orthopedic precast 700 shown in FIG. 7A, following heating at or above the lower range of the melting point of the thermoplastic material 725 of the second knitted portion 720. Upon the application of heat at or above the lower range of the melting point of the thermoplastic material 725, one or more portions 740 of the thermoplastic material 725 partially melts and diffuses to contact and engage with the first material 715. The heat may be provided by a heat source 730, which should be interpreted as any suitable heat source, including but not limited to a thermal generator or a steam generator. After cooling, the thermoplastic material 725 solidifies and the second knitted portion 720 becomes rigid while the first material 715 retains the same phase and operates as a soft interface between the rigid second portion 720 and a skin of the patient.

FIG. 8A generally depicts a schematic of a portion of an orthopedic precast 800 having a second knitted portion 822 configured with a higher concentration of a thermoplastic material 821 than a first knitted portion 810. According to this embodiment of the disclosure, upon applying heat at or above the lower range of the melting point of the thermoplastic material 821, both the first knitted portion 810 and the second knitted portion 822 may experience a partial phase change caused by partially melting of the thermoplastic material 821. As shown, the second knitted portion 822 may experience a greater volume of melted thermoplastic material 821 due to its higher concentration level. Therefore, after cooling, both the first knitted portion 810 and the second knitted portion 822 may solidify, with the second knitted portion 822 becoming more rigid than the first knitted portion 810.

As a result, the orthosis produced by the precast 800 may feature different layers with an outer layer associated with the second knitted portion 822 having a higher level of rigidity than an inner layer associated with the first knitted portion 810.

FIG. 8B generally depicts a schematic of a portion of an orthopedic precast 804 having a first knitted portion 814 configured with a first knitting pattern 815 and a second knitted portion 824 configured with a second knitting pattern 825 different from the first knitted portion 814. Herein, for this illustrative example, the second knitting pattern 825 of a thermoplastic material is tighter than the first knitting pattern 815 of the thermoplastic material. According to this precast architecture, upon applying heat at or above the lower range of the melting point of the thermoplastic material, both the first knitted portion 814 and the second knitted portion 824 may experience a partial phased change caused by partially melting of the thermoplastic material. However, given this second knitting pattern 825 features a greater amount of thermoplastic material over a prescribed distance or area than the first knitting pattern 815, the second knitted portion 824 may experience a greater volume of melted thermoplastic material. As a result, after cooling, the second knitted portion 824 may be formed as a shell portion with greater rigidity and the first knitted portion 814 may also be formed as a shell portion. Hence, different types of knitting patterns may be used to influence the rigidity of a resultant shell portion(s) of an orthosis.

FIG. 8C generally depicts a schematic of a portion of an orthopedic precast 806 having a first knitted portion 816 configured with thinner strand(s) 817 of a thermoplastic material than the second knitted portion 826. Stated differently, the second knitted portion 826 is configured with thicker strand(s) 827 of the thermoplastic material than the strand(s) 817 of the thermoplastic material in the first knitted portion 816.

According to this precast architecture, where the same thermoplastic material is utilized in the first knitted portion 816 and the second knitted portion 826, upon applying heat at or above the lower range of the melting point of the thermoplastic material, both the first knitted portion 816 and the second knitted portion 826 may experience a partial phase change caused by partially melting of the thermoplastic material. However, given that the strand(s) 827 of the thermoplastic material within the second knitting pattern 826 is(are) thicker than the strand(s) 817 of the thermoplastic material within the first knitting pattern 816, during a melting process, a greater amount of thermoplastic material within the second knitted portion 826 may be melted.

As a result, after cooling, the second knitted portion 826 would be formed as a shell portion with greater rigidity than a shell portion formed by the first knitted portion 816. Hence, different types of strand thicknesses may be used to influence the rigidity of a resultant shell portion of an orthosis.

FIG. 8D is a schematic of a portion of an orthopedic precast 808 having a second knitted portion 828 configured with a plurality of layers that include filaments of a thermoplastic material, where the number of layers of the second knitted portion 828 exceed a first knitted portion 818. According to this precast architecture, upon applying heat at or above the lower range of the melting point of the thermoplastic material, the second knitted portion 828 would experience a phase change caused by partially melting of the thermoplastic material. However, given this second knitted portion 828 has a greater amount of thermoplastic material over a prescribed distance or area than the first knitted portion 818 and the transfer of heat may be diminished as heat transfers into the first knitted portion 818 via the second knitted portion 828, the second knitted portion 828 may experience a greater volume of melted thermoplastic material. As a result, after cooling, the second knitted portion 828 would be formed as a shell portion with greater rigidity and the first knitted portion 818. Hence, multiple layers of thermoplastic material may be used to influence the rigidity of a resultant shell portion of an orthosis.

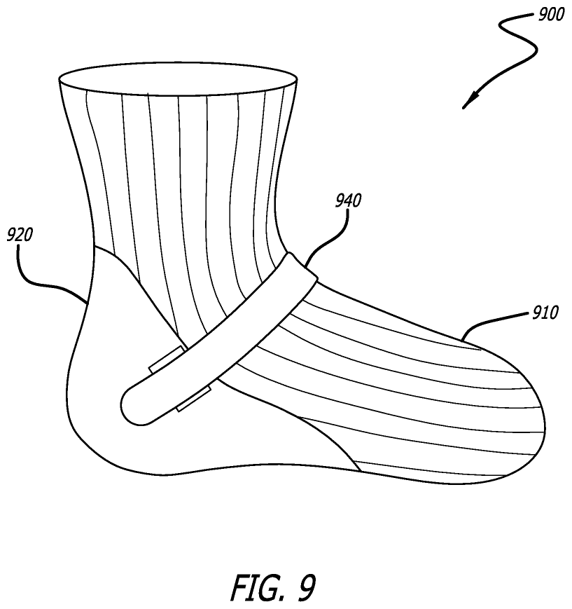

FIG. 9 generally depicts a schematic of a portion of an orthopedic precast 900 having a second knitted portion 920 that includes a thermoplastic material with a lower melting point than a first knitted portion 910. Herein, the second knitted portion 920 also includes a material other than the thermoplastic material, which increases rigidity. Contemplated rigidity increasing materials include a carbon, glass or other rigid fibers. The first portion 910 includes Kevlar™ or other material having both high levels of strength and flexibility. A strap 940, knitted in combination with the first knitted portion 910 and the second knitted portion 920, is used to assist in retaining the orthopedic precast 900 on a lower limb and foot of a patient (not shown).

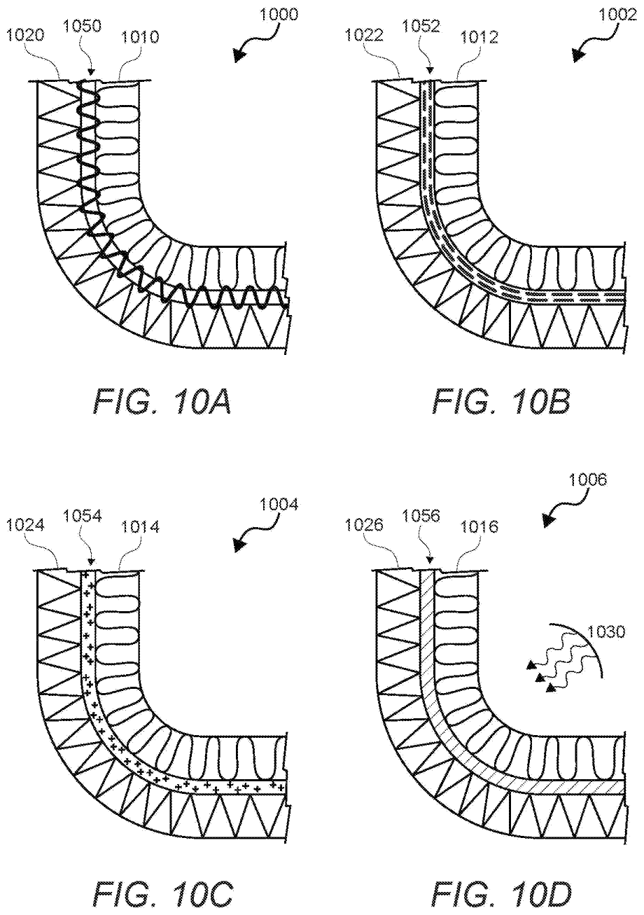

FIG. 10A generally depicts a schematic of a portion of an orthopedic precast 1000 having a second knitted portion 1020 that includes a thermoplastic material with a lower melting point than a first knitted portion 1010, in which the second portion 1020 is sewn or knitted 1050 to the first knitted portion 1010. This precast layering scheme may be utilized where the material associated with the second knitted portion 1020, after applying heat and cooling, would fail to adhere to the material associated with the first knitted portion 1010. The knitting 1050 attaches the first knitted portion 1010 and the second knitted portion 1020, where the thermoplastic material within the first or second knitted portions 1010 or 1020 fails to provide sufficient adhesion upon heat being applied within a lower range of the melting point of the thermoplastic material within the second knitted portion 1020 to melt and harden after cooling.

FIG. 10B generally depicts a schematic of a portion of an orthopedic precast 1002 having a second knitted portion 1022 that includes a thermoplastic material with a lower melting point than a first knitted portion 1012, in which the second knitted portion 1022 may be laminated to the second knitted portion 1012 at lamination region 1052. This precast layering scheme may be utilized where the material associated with the second knitted portion 1022, after applying heat to melt the thermoplastic material and subsequent cooling, would fail to adhere to the material associated with the first knitted portion 1012.

FIG. 10C generally depicts a schematic of a portion of an orthopedic precast 1004 having a second knitted portion 1024 that includes a thermoplastic material with a lower melting point than a first knitted portion 1014, in which the second knitted portion 1024 is chemical bonded to the first knitted portion 1014 at chemical bonding region 1054. This precast layering scheme may be utilized where the material associated with the second knitted portion 1024, after applying heat to melt the thermoplastic material of the second knitted portion 1024 and subsequent cooling, would fail to adhere to the material associated with the first knitted portion 1014.

FIG. 10D generally depicts a schematic of a portion of an orthopedic precast 1006 having a second knitted portion 1026 that includes a thermoplastic material with a lower melting point than a first knitted portion 1016, in which the second knitted portion 1026 is melted to the first portion 1016 at melting region 1056, using heat provided by heat source 1030. This precast layering scheme may be utilized where the material associated with the second knitted portion 1026, after applying heat to melt the thermoplastic material and subsequent cooling, normally adhere to the material associated with the first knitted portion 1016. For example, the first knitted portion 1016 may include a first type of thermoplastic material (e.g., polyethylene (PE)) while the second knitted portion 1026 may include a second type of thermoplastic material (e.g., polypropylene (PP)). The melting region 1056 between the first knitted portion 1016 and the second knitted portion 1026 is formed where PE would adhere to PP.

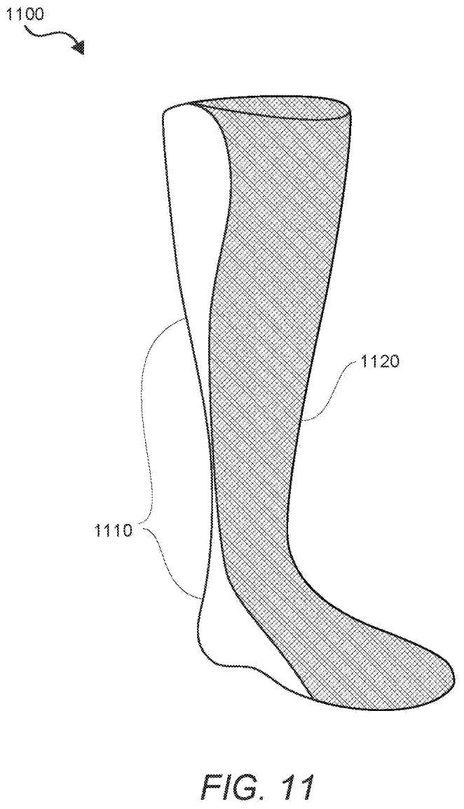

FIG. 11 generally depicts a perspective view of a portion of an orthopedic precast 1100 having a first knitted portion 1110 that includes a thermoplastic material with a lower melting point than material within a second knitted portion 1120. Herein, the second knitted portion 1120 is elastic. Elasticity can be accomplished via us of elastic threads and/or use of one or more knitting patterns that confer elasticity. Hence, while the first knitted portion 1110 and the second knitted portion 1120 are formulated concurrently, the materials forming the first knitted portion 1110 are different from the second knitted portion 1120.

Herein, after placement on the positive mold (or leg of a patient), the precast 1100 may be heated at or above the lower range of the melting point of the thermoplastic material within the first knitted portion 1110. Thereafter, after cooling, the first knitted portion 1110 is rigid to provide posterior leg stability of the patient when worn while the second knitted portion 1120 remains elastic to provide comfort and greater maneuverability by the patient.

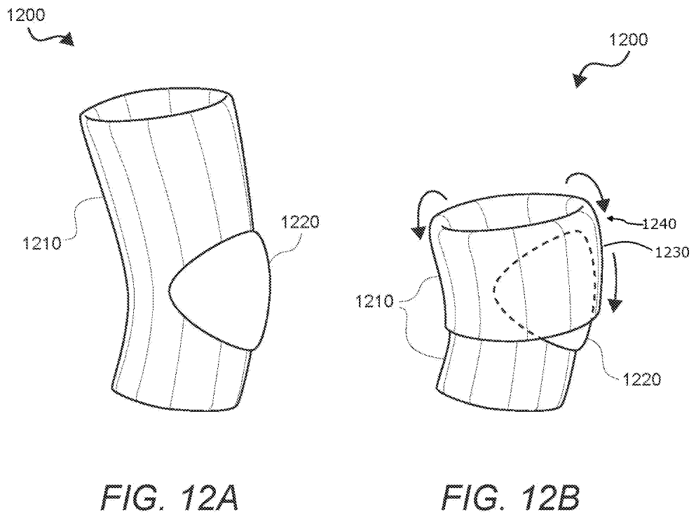

FIG. 12A generally depicts a perspective view of an orthopedic precast sleeve 1200 having a first knitted portion 1220 that includes a thermoplastic material with a lower melting point than material forming a second knitted portion 1210. Herein, the first knitted portion 1220 may be integrated at selected regions of a single knitted composite layer, which includes the first knitted portion 1220 and the second knitted portion 1210. As shown, the first knitted portion 1220 may be located one or more desired regions, such as a patella region for example.

As a result, after a prescribed amount of heat at or above a lower range of the melting point of the thermoplastic material within the first knitted portion 1220 of the precast sleeve and subsequent cooling, the first knitted portion 1220 transitions to a rigid shell portion formed to prevent the patella from being misaligned and protect the patella from blunt forces. For this example, the sleeve precast 1200 may be transformed into a customized orthosis and knee protector featuring a protective shell portion integrated as part of the single knitted composite layer.

FIG. 12B generally depicts a perspective view of the orthopedic precast sleeve 1200 shown in FIG. 12A, in which a top edge section 1230 of the precast sleeve 1200 has been folded down at least over part of the first knitted portion 1220. According to this embodiment of the disclosure, the top edge section 1230 may partially or entirely cover the first knitted portion 1220. This folding occurs prior to the heating process to convert the precast sleeve 1200 into an orthosis for use by a patient with a thicker top region 1240 to assist in donning and/or removing the orthosis, as needed. Also, where the second knitted portion 1210 includes thermoplastic material, a higher heat may applied to transition this section of the precast sleeve 1200 into a thicker, rigid shell portion to provide further patella protection.

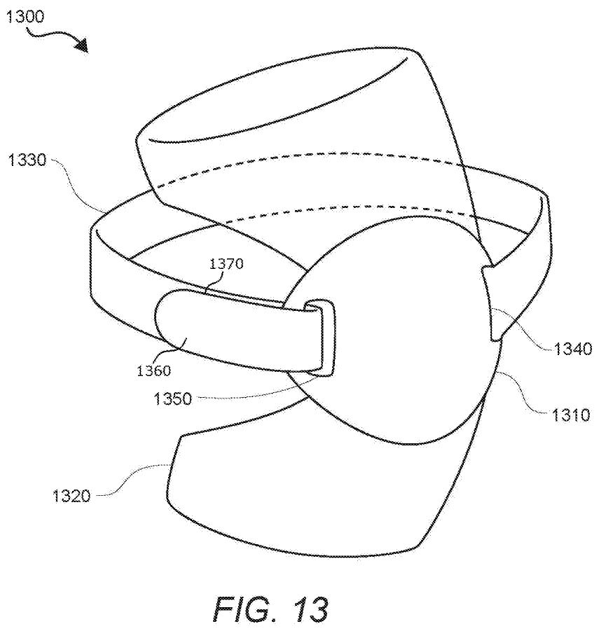

FIG. 13 generally depicts a perspective view of an orthopedic precast 1300 featuring at least a first knitted portion 1310, a second knitted portion 1320 and a third knitted portion (not shown) positioned at selected locations of the first knitted portion 1310. Herein, the third knitted portion would feature a material with a melting point lower than thermoplastic material included within the first knitted portion 1310. The first knitted portion 1310 includes the thermoplastic material with a lower melting point than material within the second knitted portion 1320.

Based on this precast structure, the heating of the orthopedic precast 1300 to a temperature at or above the lower range of the melting point of the thermoplastic material included within the first knitted portion 1310 would cause the following: (a) at least partial melting of the thermoplastic material within the first knitted portion 1310, and (b) complete melting (or incineration) of the third knitted portion to create an aperture (eye) 1350. As a result, a framework for a removable knee/elbow protective orthosis is produced from the precast 1300, where the first knitted portion 1310 transitions into a rigid shell portion and the second knitted portion 1320 may remain in a flexible or even elastic construction.

Thereafter, a strap 1330 may be included as a fourth knitting portion anchored to a footing region 1340 at the first knitted portion 1310 or may be attached as a post-production element by coupling a first end of the strap 1330 to the footing region 1340 and looping around the second end 1360 of the strap 1330 for insertion through the aperture 1350. The second end of the strap 1360 may include a fastener (e.g., a hook fastener of a hook & loop fastening mechanism) while an outer surface 1370 of the strap 1330 may include a complementary fastener (e.g., unbroken loop (UBL) material for the hook & loop fastening mechanism).

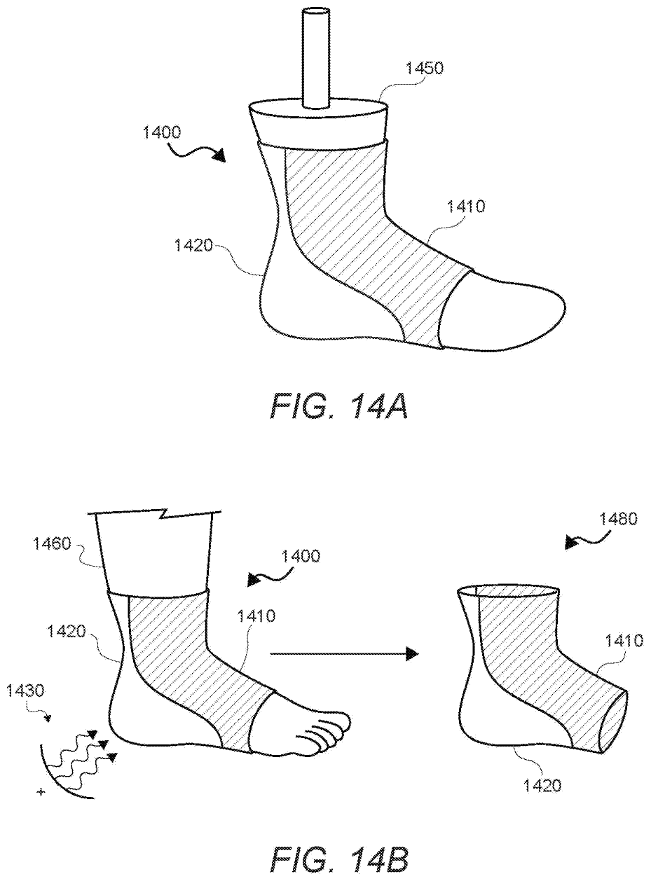

FIG. 14A generally depicts a perspective view of an orthopedic precast 1400 having a first knitted portion 1420 that includes a thermoplastic material with a lower (first) melting point than a second portion 1410. The precast 1400 is positioned about a mold 1450 of a patient's body part (e.g., foot and ankle region) to illustrate that the first knitted portion 1420 is positioned along a posterior side to cover a heel, ankle and Achilles tendon region of the patient. As described above, there are a number of advantage of utilization of the mold 1450 in creation of the orthosis 1480 of FIG. 14B. By allowing the mold 1450 to represent the patient's anatomical shape with certain relief areas (e.g., added or removed contours to relieve pressure caused by a bony prominence, added or removed contours to better direct additional load to soft tissue area for enhanced comfort, etc.), the orthosis 1480 may be better fitted to the patient.

As shown in more detail in FIG. 14B, the precast 1400 is positioned about a mold 1460 (like mold 1450) of a patient's body part (e.g., foot and ankle region), the precast 1400 is heated using a heat source 1430 (e.g., thermal heat, steam heat, etc.) to a temperature at or above the lower range of the melting point of the thermoplastic material within the first knitted portion 1420. According to one embodiment, the heating occurs for the entire precast 1400. According to another embodiment, the heating may be “spot” heating to heat targeted areas of the precast 1400 more than others. After the heating process, the precast 1400 is cooled, which at least the first knitted portion 1420 partially rigidifies into a shell portion while the second portion 1410 retains its elasticity (or at least non-rigid nature). As a result, the AFO orthosis 1480 is produced.

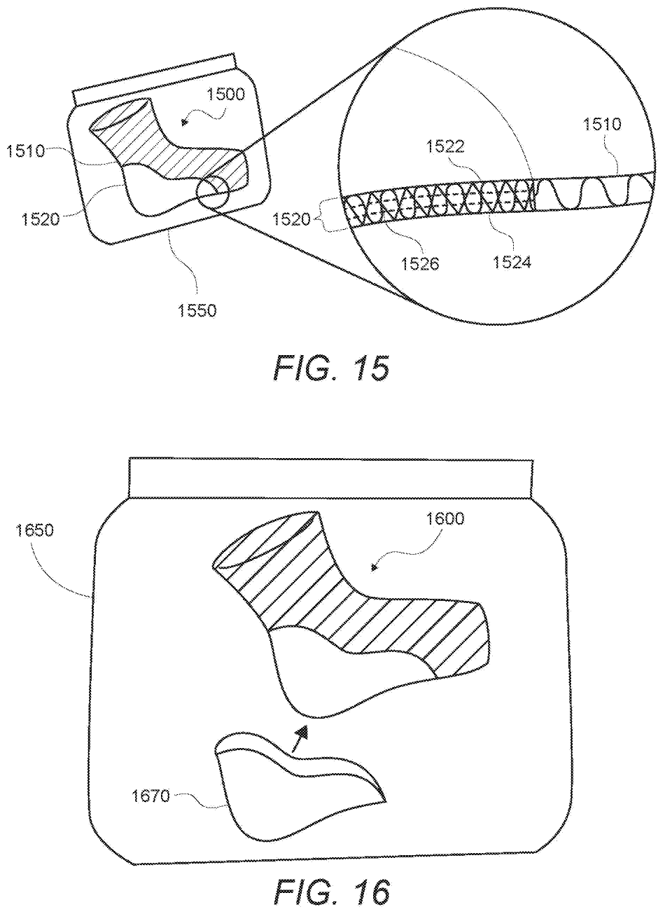

FIG. 15 generally depicts a perspective view of a self-heating, orthopedic precast 1500 disposed in a nitrogen filled bag 1550. The precast 1500 has a shell portion 1520 with thermoplastic material, which may include thermoplastic threads or yarns 1522, non-thermoplastic fibers 1524, and an amount of an embedded self-heating composition 1526. The embedded self-heating composition 1526 should be interpreted as any one or more of loose powder or other particles, particles bound to one or both of the thermoplastic threads or yarns 1522, and non-thermoplastic threads or yarns 1524 of the self-heating composition 1526. This particular example orthopedic precast 1500 is an AFO, in which the shell portion 1520 is knitted to a second, flexible portion 1510.

Any suitably functional material(s) can be employed as the embedded self-heating composition 1526, including for example, magnesium metal powder, alloyed with a small amount of iron, such as that used in heating meals-ready-to-eat (MREs). Typically such materials generate heat during an exothermic chemical reaction when triggered by oxygen in the atmosphere, and in FIG. 15, the embedded self-heating composition 1526 is prevented from doing so by being stored in a nitrogen-filled bag. In other contemplated embodiments, a suitable exothermic chemical reaction could be triggered in some other manner, such as by ambient heat or other light.

Upon removal of precast 1500 from the bag 1550, the precast 1500 is placed on a human limb or other mold, where the embedded self-heating composition 1526 comes in contact with oxygen in the air, and a chemical reaction heats the thermoplastic threads or yarns 1522 to around the melting point. Upon cooling, the thermoplastic threads or yarns 1522 partially melt together to transform the hardened shell from portion 1520.

FIG. 16 generally depicts a perspective exploded view of an alternative self-heating, orthopedic precast 1600, similar to that in FIG. 15, except that here the self-heating composition is contained in an outer cover or blanket 1670, which is removable from the precast 1600.

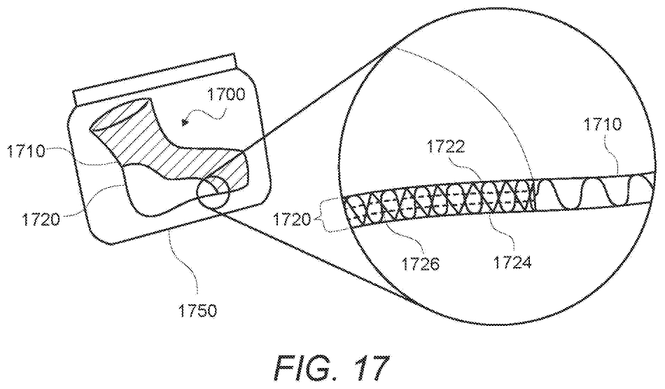

FIG. 17 generally depicts a perspective exploded view of yet another alternative orthopedic precast 1700, generally having a shell portion 1720 and a flexible portion 1710. The shell portion 1720 includes an amount of a polymerizable composition 1726, which should be interpreted as any one or more of loose powder or other particles 1726, threads or yarns 1722 that include polymerizable material, or particles of polymerizable material bound to any of the other threads or yarns 1724.

The precast 1700 is stored in bag 1750, which excludes a polymerizing agent. Contemplated polymerizing agents include, for example, ultra-violet (UV) or other light, one or more chemicals, or other suitable energy source. Upon opening of bag 1750, precast 1700 is placed on a human limb or other mold, and subject to the polymerizing agent to polymerize the polymerizable material 1726 to form a hardened shell from portion 1720.

III. Additional Illustrative Applications

The precast formulations described above are illustrative, as the invention may be expanded to a wide variety of orthoses. Herein, the orthopedic precast includes (i) a first knitted (shell) portion that features one or more strands of a first thermoplastic material and (ii) a second knitted portion mechanically coupled to the first knitted portion so that the first and second knitted portions are formed on a single knit layer. The first thermoplastic material has a melting point that would be lower than the melting point of the material utilized in the second knitted portion. Herein, upon applying heat to the orthopedic precast at or above the lower range of the melting point of the first thermoplastic material, the first knitted portion transitions to a rigid shell portion while the second knitted portion remains flexible and, in some case, elastic.

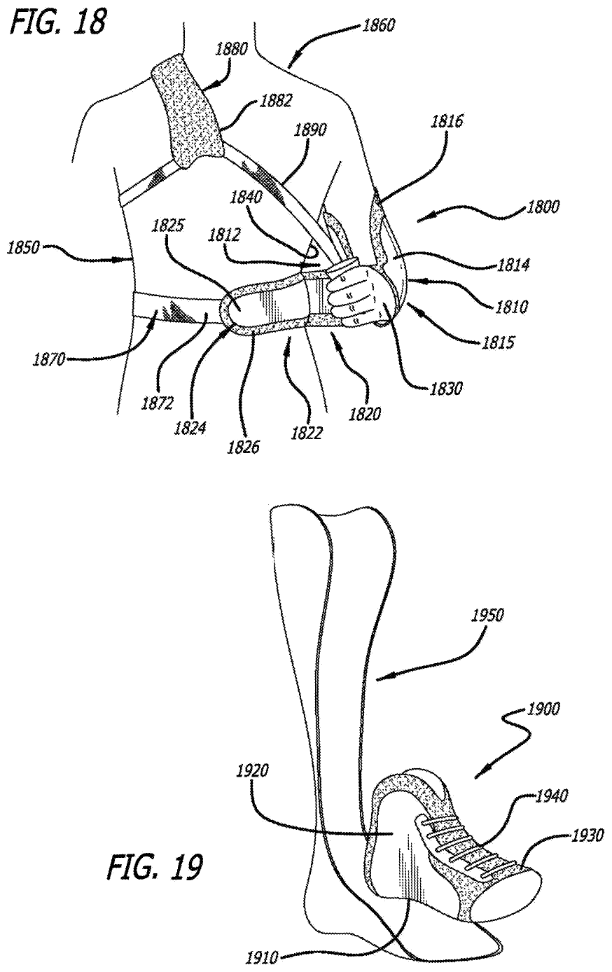

Referring to FIG. 18, an orthopedic precast (as described above) may be applicable to a shoulder orthosis 1800 including the first knitted (shell) portion and a second knitted (flexible) portion. Herein, the shoulder orthosis 1800 may be provided as an arm sling 1810 and a shoulder adjustment member 1820 that maintains a patient's arm 1830 within the arm sling 1810 at a prescribed distance from a side 1840 of the patient 1850. The shoulder adjustment member 1820 is positioned generally orthogonal to an inner forearm area 1812 of the arm sling 1810 to position an elbow and forearm regions in order to position a shoulder 1860 of the patient 1850. The hip shell 1825 and belt, when connected through a rigid structure of the first knitted (shell) portion at the forearm creates abduction and rotational control of the shoulder 1860.

According to one embodiment of the shoulder orthosis 1800, as shown in FIG. 18, a first end (not shown) of the shoulder adjustment member 1820 is mechanically coupled the inner forearm area 1812 of the arm sling 1810 while a second end 1822 of the shoulder adjustment member 1820 includes at least a first attachment member 1824 for coupling to a belt 1870 extending around a waist of the patient 1850. The first attachment member 1824 may include a loop fastener for mating with an unbroken loop (UBL) connector positioned on a top surface 1872 of the belt 1870.

The arm sling 1810 includes a first knitted portion 1814 positioned around the forearm/elbow area 1812/1815 and surrounding both sides of the patient's forearm to immobilize the arm 1830 and create abduction and rotational control of the shoulder 1860. The arm sling 1810 further includes a second knitted portion 1816 surrounding a portion of the perimeter of the arm sling 1810 to provide a soft transition to protect the patient from being cut or pinched by slight movement of the first knitted portion 1814. When heated, the first knitted portion 1814 becomes rigid similar to a cast while the second knitted portion 1816 retains its flexibility. Herein the first knitted portion 1814 includes thermoplastic material in an amount greatly exceeding any thermoplastic material, if any, included as part of the second knitted portion 1816. Similarly, the shoulder adjustment member 1820 includes at least the first attachment member 1824 includes a first knitted portion 1825 positioned along a middle section of the first attachment member 1824 and a second knitted portion 1826 positioned along a perimeter of the first knitted portion 1825. As before, this concentration provides sufficient forces to restrict lateral movement of the arm sling 1810.

As further shown in FIG. 18, a shoulder pad 1880 is formed of a second material which may include flexible, smooth and/or elastic material to avoid chafing of the patient's neck and/or shoulder area due to use. For example, the second material may be a soft thermoplastic material such as polyethylene (PE). Herein, where the second material has a higher melting point range that material associated with the first knitted portion 1814/1825, the shoulder pad 1880 may retain its flexible and/or elastic construction. The shoulder pad 1880 has a looped edge 1882 to allow a strap 1890 to feed there through to vertically support the arm sling 1810.

As yet another alternative, although not shown, the shoulder pad 1880 may be molded and hardened to provide elevated support to the arm sling 1810 via the strap 1890.

A similar construction may be utilized for clothing outside of orthosis design. For example, as shown in FIG. 19, a shoe 1900 (e.g., slipper, post-operative shoe as shown, etc.) may be configured with the first knitted (shell) portion positioned along a foot plate 1910 and selected areas surrounding an ankle area 1920. When heated, the foot plate 1910 and portions of the ankle area become rigid to immobilize and protect the patient's foot. The remainder of the shoe (e.g., second knitted portion 1930) features soft fabric, padding and/or other material such as laces 1940 to provide lateral tightening of the shoe 1900. The post-operative shoe 1900 may be used in combination with a rear leg stabilizer 1950 made from a precast of the first knitted (shell) material (or a version thereof) and shaped around a posterior of a patient's leg as shown, a leg-ankle-foot orthosis (AFO) 200 as illustrated in FIG. 2, or may be used independently.

With respect to clothing, although not shown, a variety of implementations may leverage the conversion of flexible strands of material in a non-heated state into a rigid area. For example, a glove may be provided in which certain areas of the glove, when heated, produce an increased rigidity to protect a wearer's hands such as a bicycle glove. A sock may be provided in which certain areas of the sock (e.g., covering a shin area) include the first knitted portion (thermoplastic strands) while the remainder of the sock includes a second knitted portion (cotton strands) so that, when heated and cooled, the first knitted portion becomes rigid to protect a wearer's shins during a sports game.

Additionally, the clothing may constitute a shirt with (i) an interior pocket surface on the shirt featuring the first knitted portion that, when hardened, protect the wear from cellular radiation and/or radiate heat emitted from a cellular phone and/or (ii) a collar featuring the first knitted portion (with thermoplastic material) to orient the collar shape as desired, which is maintained even after laundry cycles in a washer and dryer (i.e., thermoplastic material melting point is substantially more than a maximum temperature of an electric or gas dryer).

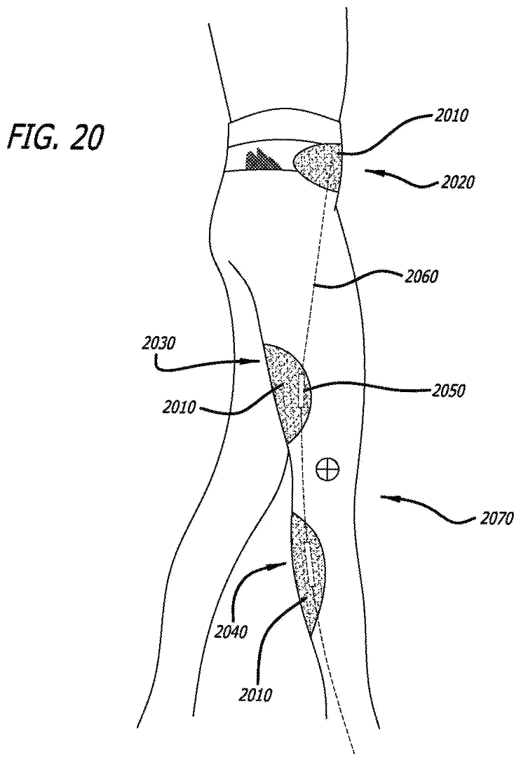

Additionally, in in the alternative, as shown in FIG. 20, the clothing may constitute pants 2000 with rigidity areas 2010 along the hips 2020, hamstring 2030 and/or calf 2040 as shown. Besides the use of stands with thermoplastic material with the first knitted portion to provide rigidity for mitigating external forces applied to certain body portions (e.g., hip, hamstring area, calf area, etc.) as shown in FIG. 20, the stands of thermoplastic material may be knitted to form one or more channels 2050 within the object (e.g., orthosis, clothing, etc.). These channel(s) 2050 may be sized for the propagation of wires, cords and/or interconnects (e.g., cable, elastic cords, etc.) 2060 within the object (e.g., pants 2000).

For example, according to one embodiment, by applying elastic tensioning of an interconnect 2060 by arranging a routing of the interconnect 2060 posterior to the knee joint, this would create a flexión moment for a knee joint 2070. Similarly, where the interconnect 2060 is routed anterior to the knee joint 2070, knee extension would be facilitated. Stated differently, the interconnect 2060 may be included within the channel(s) 2050 between two regions, where directional forces could be created between these two rigid regions. As shown in FIG. 20, with one rigid region positioned to cover a patient's thigh area and one rigid region positioned to cover the patient's calf, the interconnect 2060 could facilitate flexión or extension of the knee joint depending on placement of the cords relative to the anatomical joint and the tension and elasticity of the interconnect 2060. The interconnect 2060 could be cables with elastic sections in series or at either end, able to be tensioned with a spool or other means.

It is contemplated that this architecture may be utilized in numerous locations: shoulder, elbow, hip, and ankle. The advantage is a single layer of material with ability to control routing of cables for tensioning/controlling motion across joints. Additionally, or in the alternative, one or more interconnects 2060 may be installed within the channel(s) 2050 to restrict motion across a joint or the interconnects 2060 or the interconnect(s) 2060 may constitute electrical wires positioned within the channel(s) 2050 to apply electromagnetic therapeutic pulses to stimulate tissue healing at various body parts.

These and other products may be formulated in accordance with selected positioning of knitted portions with thermoplastic materials, heating, and subsequent cooling to achieve rigidity of these knitted portions.

It is still further contemplated instead of thermoplastic strands (e.g., threads, yarns, etc.) being heated and then cooled to form a shell portion, material could be used in the strands that is hardened by polymerization, with the polymerizing energy coming from ambient or artificial light, an oxidizing or reducing agent, or any other suitable energy source.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. A knitted wearable, comprising:
a first knitted portion comprising knitted strands that includes at least a thermoplastic material that at least partially melts at a first melting point; and
a second knitted portion mechanically coupled to the first knitted portion,
wherein the first knitted portion is configured such that, following heating of the knitted product to at least the first melting point, and subsequent cooling, the first knitted portion is more rigid in at least one dimension than the second knitted portion.

2. The knitted wearable of claim 1, wherein the first knitted portion is configured with a higher concentration of the thermoplastic material than in the second knitted portion.

3. The knitted wearable of claim 1, wherein the first knitted portion is configured with a tighter knit than in the second knitted portion.

4. The knitted wearable of claim 1, wherein the first knitted portion is configured with thicker filaments of the thermoplastic material than in the second knitted portion.

5. The knitted wearable of claim 1, wherein the first knitted portion is configured with a greater number of layers of material than in a number of layers of material utilized by the second knitted portion.

6. The knitted wearable of claim 1, wherein the first knitted portion is configured with a plurality of different types of thermoplastic material to increases rigidity beyond use of a single type of thermoplastic material.

7. The knitted wearable of claim 1, wherein the second knitted portion includes strands of non-thermoplastic material to provide elasticity of the second knitted portion while the first knitted portion remains rigid.

8. The knitted wearable of claim 1, wherein the first knitted portion is sewn or knitted to the second knitted portion.

9. The knitted wearable of claim 1, wherein the first knitted portion is laminated to the second knitted portion.

10. The knitted wearable of claim 1, wherein the first knitted portion is chemically bonded or fused to the second knitted portion.

11. The knitted wearable of claim 1, wherein the first knitted portion features a first knitting pattern different than a second knitting pattern where the knitted strands of the thermoplastic material knitted in accordance with the first knitting pattern is more rigid than the knitted strands of the thermoplastic material knitted in accordance with the second knitting pattern.

12. The knitted wearable of claim 1, further comprising a tube that includes both the first knitted portion and the second knitted portion.

13. The knitted wearable of claim 1, wherein the thermoplastic material comprises at least eight percent weight of the first knitted portion.

14. The knitted wearable of claim 13, wherein the first knitted portion comprises at least eight percent of an overall weight of the precast.

15. The knitted wearable of claim 1, wherein the thermoplastic material is resilient with respect to bending following partially melting and cooling to room temperature.

16. The knitted wearable of claim 1, wherein the second knitted portion includes at least a second thermoplastic material having a higher melting temperature than the first melting point and is physically separated from the first knitted portion.

17. The knitted wearable of claim 16, wherein the second knitted portion includes non-thermoplastic material in combination with the second thermoplastic material.

18. The knitted wearable of claim 1, wherein the knitted strands of the first knitted portion are a composite including at least strands of the thermoplastic material and strands of another type of material.

19. The knitted wearable of claim 18, wherein the strands of another type of material include non-thermoplastic material have melting points that differ by at least 50° Celsius from the thermoplastic material and a volume of the thermoplastic material is greater than a volume of the non-thermoplastic material.

20. The knitted wearable of claim 1, wherein the first knitted portion and the second knitted portion reside within a same layer of knitted material forming the precast.

21. The knitted wearable of claim 1, wherein the second knitted portion is layered with the first knitted portion and is placed into a different layer of knitted material than the first knitted portion.

22. The knitted wearable of claim 1 further comprising a third knitted material with a melting point less than the first melting point so that, when the precast is heated to the first melting point, the third knitted material is removed leaving at least one of an eye or a footing for a strap positioned within the first knitted portion.

23. The knitted wearable of claim 1 being an orthopedic precast.

24. The knitted wearable of claim 1 being a clothing item such as a sock.

25. A method of producing a custom orthosis for a patient, comprising:
placing an orthopedic precast about a mold, the precast including a shell portion including first knitted strands that include at least a first thermoplastic material that at least partially melts at a first melting point, and a second portion including a second knitted strands with a second melting point significantly greater than the first melting point so that the first knitted strands can change phase and melt without changing a phase of the second knitted strands; and
heating and then cooling the precast to at least the first melting point to at least partially melt and rigidify the shell portion.

26. The method of claim 25, wherein the precast comprises a tube that includes both the shell portion and the second portion, and further comprising pulling the tube over the mold.

27. The method of claim 25, wherein the mold is a positive mold.

28. The method of claim 25, wherein
the placing an orthopedic precast including forming the precast in which the shell portion further includes third knitted strands of a second thermoplastic material having a lower melting point than the first thermoplastic material;
the heating and then cooling of the precast comprises applying a temperature to the precast that melts at least some of the second thermoplastic material, but refrains from melting the first thermoplastic material.

29. The method of claim 25, further comprising selecting the first thermoplastic material such that the shell portion is resilient to bending.

30. A method of producing an orthosis, comprising:
removing an orthopedic precast from a container, the precast comprising (i) a first knitted portion that includes a material configured to harden upon application of a hardening agent, and (ii) a second knitted portion that remains flexible upon application of the hardening agent;
placing the precast about a human limb or other mold;
applying the hardening agent to the precast to form an orthosis with a hardened portion by transformation of the first knitted portion and a flexible portion by transformation of the second knitted portion; and
removing the orthosis produced from the orthopedic precast from the mold.

31. The method of claim 30, wherein the material configured to harden comprises strands of thermoplastic material and the hardening agent comprises an application of heat.

32. The method of claim 30, wherein the material configured to harden comprises a self-heating composition including strands of thermoplastic material that, upon exposure to ambient oxygen, causes the self-heating composition to at least partially melt.

33. The method of claim 30, wherein the material configured to harden includes a polymerizable composition, the hardening agent comprises a polymerizing agent, and further comprising applying the polymerizing agent sufficient to polymerize the polymerizable composition.
